(19) United States
(12) Patent Application Publication
(10) Pub. No.: US 2007/0037717 A1
(43) Pub. Date: Feb. 15, 2007

(54) Denture adhesive solvent compositions

(75) Inventors: Hal C. Clark, Parsippany, NJ (US); Stanley J. Lech, Parsippany, NJ (US); Naresh I. Mehta, Pasippany, NJ (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philadelphia, PA (US)

(21) Appl. No.: 10/572,969

(22) PCT Filed: Oct. 6, 2004
(86) PCT No.: PCT/US04/32829

§ 371 Date: Mar. 21, 2006

Related U.S. Application Data
(60) Provisional application No. 60/508,895, filed on Oct. 6, 2003.

(51) Int. Cl. A61K 8/00

(52) U.S. Cl. 510/116

(57) ABSTRACT

Denture adhesive solvent compositions comprising at least one oil based solvent, a thickener and a surfactant suitable for removing denture adhesive and adhesive residue from the denture and hard and soft tissues of the oral cavity and denture adhesive removal systems comprising a wipe comprising a flexible substrate premoistened with a denture adhesive solvent composition are disclosed.

### FIELD OF THE INVENTION

The present invention relates to denture adhesive solvent compositions useful for the removal of denture adhesive and adhesive residue from dentures and from the natural surfaces of the oral cavity. The present invention also relates to a denture adhesive removal system comprising a denture cleansing wipe premoistened with a denture adhesive solvent composition.

### BACKGROUND OF THE INVENTION

Dentures and dental plates function as a substitute for all or part of missing teeth ordinarily found in the mouth. While dentures are usually carefully fitted for the user, the fit can change over time, due to natural shrinkage and changes in the gum or mucosal tissue, causing discomfort and slippage. To alleviate the discomfort and to control the slippage, a denture adhesive may be applied to the denture to fill the interstices between the dentures and the gum or tissues. Sometimes referred to as a denture stabilizer, the denture adhesive is formulated not only for its adherent properties, but also to provide a cushion or gasket between the denture and the gums or tissues, thereby positioning the denture securely in the oral cavity.

Common forms of the denture adhesive, or stabilizer, include denture adhesive creams, powders, gels, pastes, liquids and liners. Typically these types of denture adhesives are intended to form a seal or gasket between the denture and the gum line. The adhesive is applied into the gum receiving plate and properly positioned in the mouth. With the application of pressure, the denture is securely locked in place. Often, the sealing or gasket-forming material includes a water swellable gum or polymer. The gum or polymer hydrates and becomes tacky when introduced to the saliva in the oral cavity, thus assisting in securing the dentures in place. Oils and petrolatum are often utilized in such adhesive compositions to suspend the gum or polymer and delay the washing away of the adhesive materials by the flow of saliva and consumed fluids.

As new formulations for denture adhesives and stronger adhesive components have been developed, the coadhesive and adhesive properties have been dramatically improved to the point where removal of the adhesive from the denture after removal from the mouth for cleaning or replacement is often very difficult and time consuming. Once the dentures are removed from the oral cavity, it may also be difficult to remove the denture adhesive material from the user's soft gum tissue and palate.

Compositions useful for the removal of denture adhesives have been identified. U.S. Pat. Nos. 4,701,223 and 4,807,649, both to Eoga, disclose gel and sprayable liquid denture cleansers comprising a water soluble detergent selected from the group consisting of sulfonated, sulfated and sulfoacetate fatty alcohols, a chelating agent, water, a gelling agent, where appropriate, and, optionally, a non-toxic glycol. U.S. Pat. No. 6,518,227 to Woosley discloses an oil-based solvent composition for denture adhesives, suitable for use in the oral cavity, comprising tea tree oil (Melaleuca Altemifolia oil) in an oil-based carrier, such as vegetable oil. However, Applicant believes that there is no effective commercial formulation available for the removal of denture adhesive from the denture and the oral cavity and, therefore, there remains a need for convenient and effective denture adhesive remover formulations and systems.

Brushes and other scraping devices that require the denture wearer to physically remove the denture adhesive from the denture by wiping and scrubbing are also known. See U.S. Pat. No. 5,032,082 to Herrara; U.S. Pat. No. 5,261,817 to Nack; and U.S. Pat. No. 5,987,689 to Gordon. Such devices may cause irritation when used directly on the soft oral tissues. Brush heads may be too large and unyielding to effectively access all areas of the denture plate. In addition, these devices can be bulky and difficult to conceal, thus, making discrete, portable and convenient removal of denture adhesive and denture adhesive residue difficult.

Wet wipes are typically premoistened, disposable towelettes which may be utilised in a variety of applications both domestic and industrial and perform a variety of functions. Wet wipes are typically used to wipe surfaces both animate and inanimate, and may provide numerous benefits such as cleaning, cleansing, disinfecting, and skin care benefits. One particular application is the use of wet wipes for wiping parts of the human body particularly when wash water is not available, for example when travelling. Wipes are commonly used for human cleansing and wiping. Wet wipes may also be used for application of substances to the body including removing and applying of make-up, skin conditioners and medications. Another application of wipes is during diaper changes and also for the treatment of adult and baby dermatitis partly caused by infrequent changing of diapers and incontinence devices. In addition wet wipes are also applicable for wiping and/or cleaning other surfaces or for the application of compositions to surfaces, for example kitchen and bathroom surfaces, eyeglasses, shoes, automobiles and surfaces which require cleaning and/or sanitizing in industry such as surfaces of machinery. Applicant believes that a wet wipe suitable for denture adhesive removal, wherein the wet wipe is premoistened with a denture adhesive solvent composition is not known.

### SUMMARY OF THE INVENTION

The present invention relates to a solvent composition, particularly useful for the removal of denture adhesive and denture adhesive residue from the denture and from the natural surfaces of the oral cavity. Denture adhesive solvent compositions of the present invention comprise at least one oil-based solvent, a surfactant and a thickener. In one embodiment the oil-based solvent comprises mineral oil. In another embodiment the oil-based solvent comprises mineral oil and vegetable oil. In yet another embodiment the denture adhesive solvent compositions further comprises a flavorant and/or a cosmetic or therapeutic active. The present invention also relates to a premoistened wet wipe suitable for denture adhesive removal wherein the wet wipe is premoistened with a denture adhesive solvent composition.

### DETAILED DESCRIPTION OF THE INVENTION

The present invention relates to a solvent composition, particularly useful for the removal of denture adhesive and denture adhesive residue from the denture and from the natural surfaces of the oral cavity.

The term “denture(s)”, as used herein, is meant to include full and partial dentures, dental plates, bridges, artificial teeth and other hard surfaces of dental appliances which are temporarily fixed within the oral cavity and which are typically removed from the oral cavity for cleaning.

The phrase “natural surfaces of the oral cavity” as used herein is meant to include teeth, gums, palate, tongue or any other natural surface of the oral cavity that may be exposed to denture adhesives.

Certain denture adhesive solvent compositions of the present invention comprise at least one oil-based solvent, a surfactant and a thickener. The oil-based solvent is typically present from about 0.1% to about 90% by weight of the composition. Suitable oil-based solvents include mineral oil, vegetable oil, D-limonene, glycerin, cottonseed oil, sesame oil, lecithin, propylene glycol and mixtures thereof. In one embodiment the oil-based solvent is mineral oil. Where the denture adhesive solvent composition comprises mineral oil, a separate carrier material may be included. Suitable carriers include, vegetable oil, D-limonene, glycerin, cottonseed oil, sesame oil, lecithin, propylene glycol, water and mixtures thereof. In one embodiment the oil-based solvent is a mixture of mineral oil at a level of from about 1% to about 90% and vegetable oil at a level of from about 1% to about 30%.

Surfactants are present in the compositions of the instant invention from about 0.1% to about 15% by weight of the composition. Suitable surfactants are those surfactants that are designed for use in oral care and denture care compositions, including but not limited to; sulfated, sulfonated and sulfoacetate fatty alcohols such as sodium lauryl sulfate and potassium lauryl sulfate; cocamidopropyl bentaine and lecithin.

Thickeners are present in the compositions of the instant invention from about 1% to about 50%. Suitable thickeners include, but are not limited to, silicas, such as the Cabosil line of silicas manufactured by Cabot Corp., the Sident line of silicas manufactured by Degussa AG, and the Zeofree line of silicas manufactured by J.M. Huber Corp. In one embodiment, where the oil-based solvent is mineral oil, a mixture of ethylene/propylene/styrene and butylene/ethylene/styrene copolymers may be incorporated as a thickener. Mineral oil gels that includes this ethylene/propylene/styrene and butylene/ethylene/styrene copolymer mixture are manufactured by Penreco, Houston, Tex. and sold under the Versagels™ M series tradename.

Flavorants or sweeteners may be incorporated in the compositions of the present invention. Flavorants and sweeteners are typically present from about 0.1% to about 5% by weight of the composition. Suitable flavorants include, but are not limited to, essential oils such as methyl salicylate, anise, anethol, bergamot, camphor, cinnaminic anhydrides, clove, eucalyptol, peppermint, spearmint, and thyme.

The compositions of the present invention may optionally further comprise one or more cosmetic or therapeutic actives where, upon directed use, the benefit sought by the wearer is promoted without detriment to the oral cavity. Examples of the conditions these actives may address include, but are not limited to, appearance and structural changes to teeth, treatment and prevention of plaque, calculus, cavities in remaining natural teeth, inflamed and/or bleeding gums, gingivitis, fungal infections such as those associated with candida albicans, mucosal wounds, lesions, ulcers, aphthous ulcers, cold sores, tooth abscesses, and the elimination of mouth malodor resulting from the conditions above and other causes such as microbial proliferation. Suitable cosmetic or therapeutic actives include any material that is generally considered safe for use in the oral cavity. The cosmetic and therapeutic actives may be selected from the group consisting of anti-calculus agents; anti-caries agents such as fluoride ion sources; stannous ion sources; tooth whitening agents; breath fresheners; anti-microbial agents; anti-plaque agents; anti-inflammatory agents; nutrients; antioxidants; anti-bacterial agents; anti-fungal agents; analgesic agents; local anesthetic agents; antihistamines; disinfectants; vasoconstrictors; hemostatics; chemotherapeutics; antibiotics; tooth desensitizing agents; antifungals; vasodilators; antihyperstensives; antiemetics; antimigraine; antiarrhythmics; antiasthmatics; antidepressants; vaccines; peptides or prodrugs; hormones; proton pump inhibitors; and H2 receptor antagonists.

Other optional ingredients that may be incorporated into the compositions of the present invention include fragrances; sensates; pigments; dyes; lakes; colorants; and mixtures thereof.

The composition of the present invention may be used alone or in combination with an applicator or other system for removing denture adhesive. In one embodiment, the denture adhesive solvent composition may be painted on with a brush or similar applicator, sprayed on with an areosol or mechanical spray applicator, or used as a spot treatment with a pen-type or similar applicator. In one embodiment a premoistened wet wipe is used in a system for denture adhesive removal.

Accordingly, this invention also related to denture adhesive removal systems comprising a premoistened wipe suitable for denture adhesive removal, wherein the wipe is premoistened with denture adhesive solvent composition. In one embodiment the denture adhesive solvent composition may comprise at least one oil-based solvent, a surfactant and a thickener as described above.

The wipe of the present invention may be any wipe known in the art that comprises a flexible substrate which is coated or impregnated with a denture adhesive solvent composition composition. The substrate may be woven or nonwoven, foam, sponge, battings, balls, puffs or films, most preferably a nonwoven and may be composed or natural or synthetic fibres or mixtures thereof. Preferably, the fibre compositions are a mixed of hydrophilic fibre material such as viscose, cotton, or flax and a hydrophobic fibre material such as polyethylene tetraphthalate (PET) or polypropylene (PP) in a ratio of 20%-80% hydrophilic and 80%-20% hydrophobic material by weight. The substrate preferably has a basis weight of at least 20 gm⁻² and preferably less than 150 gm⁻², and most preferably the base weight is in the range of 20 gm⁻² to 70 gm⁻², more preferably from 50 gm⁻² to 65 gm⁻². The substrate may have any thickness suitable for the intended use. Examples of premoistened wipes which comprise substrates suitable for use in the present invention include, but are not limited to, those manufactured by the Procter & Gamble Company and sold as Pampers® Big Wipes, Wet Ones® manufactured by Playtex Products, Inc., and those sold by SmithKline Beecham Consumer Healthcare under the tradename Oxy®).

The wet wipes may then be individually wrapped, provided in a folded stacked configuration within a covered container such as a tub-like container having a lid. Alternatively, the wipes may be provided in tub or cylindrical container having a dispensing aperture. In each case this allows easy transportation and storage of the wipes.

Various wet wipes, which comprise flexible substrates that may be suitable for use in the denture adhesive removal systems of the present invention are described in more detail in the following U.S. Pat. Nos. 4,896,768; 6,413,599; 6,550,634; 6,602,955; all of which are incorporated herein by reference in their entirety.

### EXAMPLES

| | | Ex. 1 | Ex. 2 | Ex. 3 | Ex. 4 | Ex. 5 | Ex. 6 |
|---|---|---|---|---|---|---|---|
| INGREDIENT | Function | wt. % | wt. % | wt. % | wt. % | wt. % | wt. % |
| Mineral oil USP | Solvent | 83.6 | 85.2 | 27.00 | 70.0 | 85.14 | 50.0 |
| Kaydol | | | | | | | |
| Cocamidopropyl | Surfactant | | 1.0 | 7.90 | | 4.1 | |
| Bentaine | | | | | | | |
| Sodium Lauryl | Surfactant | 1.5 | 1.0 | 2.50 | 5.0 | | 1.0 |
| Sulfate | | | | | | | |
| Water | Solvent | | | 39.30 | | | |
| Silicate - | Thickener | | | 23.00 | | | 48.0 |
| Sident-22 | | | | | | | |
| Flavor | | 0.9 | | 0.30 | | | 1.0 |
| Silicate - Zeofree | Thickener | 14.0 | 5.1 | | 14.0 | 10.25 | |
| 153 | | | | | | | |
| Vegetable oil | Solvent | | | | 11.0 | | |
| (Sun flower oil) | | | | | | | |
| Menthol | Flavorant | | | | | 0.51 | |
| Lecithin | (Solvent) | | 7.7 | | | | |
| | Surfactant | | | | | | |

The above formulations may be prepared by the following steps: charge the mineral oil to a 1000 milliliter beaker; blend the mineral oil with a suitable mixer, such as a Lighting mixer; add the thickener component and blend until evenly mixed; add the surfactant and any flavorant or other optional ingredients; continue to mix the composition while heating the composition to about 55° C. and until the components are thoroughly blended.

A panel of denture wearers evaluated the senture adhesive remover composition of the present invention. Super PoliGrip denture adhesive was applied to lab dentures. The dentures were soaked for one to two hours to hydrate the adhesive. Each subject was given one denture and a sample of the composition.

Subjects were asked to remove the adhesive from the dentures by wiping after applying the composition to the adhesive with a brush applicator. Denture wearers indicated their current method of denture adhesive removal and compared the current method to the composition of the present invention.

| | Number of |
|---|---|
| Current Method of Adhesive Removal | Denture Wearers |
| brush denture with toothbrush under water | 5 |
| wash denture with water | 4 |
| brush denture with toothpaste | 2 |
| soak denture in water, subsequent brushing | 1 |
| soak denture in water only | 1 |
| brush denture under hot water, subsequent wiping with | 1 |
| paper towel | |
| use brush or paper towel to remove adhesive | 1 |

Of the denture wearers studied, 60% indicated that the ease of using the product was 60% better than their current method of adhesive removal. Two thirds of the denture wearers indicated that the composition provided for faster removal of the denture adhesive than their current method.

1. A denture adhesive solvent composition comprising:
a.) at least one oil-based solvent;
b.) a thickener; and
c.) a surfactant.

2. The denture adhesive solvent composition of claim 1 wherein the oil-based solvent is selected from the group consisting of mineral oil, vegetable oil, D-limonene, glycerin, cottonseed oil, sesame oil, propylene glycol and mixtures thereof.

3. The denture adhesive solvent composition of claim 2 wherein the oil-based solvent is mineral oil.

4. The denture adhesive solvent composition of claim 3 wherein the mineral oil is present from about 1% to about 90%, by weight of the composition.

5. The denture adhesive solvent composition of claim 2 wherein the oil-based solvent is a mixture of mineral oil and vegetable oil.

6. The denture adhesive solvent composition of claim 5 wherein the mineral oil is present from about 1% to about 90% and the vegetable oil is present from about 1% to about 30%, by weight of the composition.

7. The denture adhesive solvent composition of claim 1 wherein the surfactant is present from about 0.1% to about 15%, by weight of the composition.

8. The denture adhesive solvent composition of claim 7 wherein the surfactant is sodium lauryl sulfate.

9. The denture adhesive solvent composition of claim 1 wherein the thickener is present from about 1% to about 50%, by weight of the composition.

10. The denture adhesive solvent composition of claim 9 wherein the thickener is silicon dioxide.

11. The denture adhesive composition of claim 4 further comprising from about 1% to about 50% of water.

12. The denture adhesive solvent composition of claim 1 further comprising a flavorant from about 0.1% to about 10% by weight of the composition.

13. The denture adhesive solvent composition of claim 1 further comprising at least one optional ingredient selected from the group consisting of anti-calculus agents; anti-caries agents such as fluoride ion sources; stannous ion sources; tooth whitening agents; breath fresheners; anti-microbial agents; anti-plaque agents; anti-inflammatory agents; nutrients; antioxidants; anti-bacterial agents; anti-fungal agents; analgesic agents; local anesthetic agents; antihistamines; disinfectants; vasoconstrictors; hemostatics; chemotherapeutics; antibiotics; tooth desensitizing agents; antifungals; vasodilators; antihyperstensives; antiemetics; antimigraine; antiarrhythmics; antiasthmatics; antidepressants; vaccines; peptides or prodrugs; hormones; proton pump inhibitors; H2 receptor antragonists; fragrances; sensates; pigments; dyes; lakes; colorants; and mixtures thereof.

14. A denture adhesive solvent composition comprising:
a.) from about 1% to about 90% mineral oil;
b.) from about 1% to about 30% of carrier;
c.) from about 0.1% to about 15% of a surfactant;
d.) from about 0.1% to about 50% of a thickener.

15. The denture adhesive solvent composition of claim 14 further comprising a flavorant from about 0.1% to about 10% by weight of the composition.

16. The denture adhesive solvent composition of claim 15 wherein the carrier is selected from the group consisting of vegetable oil, D-limonene, glycerin, cottonseed oil, sesame oil, propylene glycol, water and mixtures thereof.

17. The denture adhesive solvent composition of claim 16 wherein the carrier is vegetable oil.

18. The denture adhesive solvent composition of claim 14 wherein the surfactant is sodium lauryl sulfate.

19. The denture adhesive solvent composition of claim 14 wherein the thickener is silicon dioxide.

20. The denture adhesive solvent composition of claim 14 further comprising at least one optional ingredient selected from the group consisting of anti-calculus agents; anti-caries agents such as fluoride ion sources; stannous ion sources; tooth whitening agents; breath fresheners; anti-microbial agents; anti-plaque agents; anti-inflammatory agents; nutrients; antioxidants; anti-bacterial agents; antifungal agents; analgesic agents; local anesthetic agents; antihistamines; disinfectants; vasoconstrictors; hemostatics; chemotherapeutics; antibiotics; tooth desensitizing agents; antifungals; vasodilators; antihyperstensives; antiemetics; antimigraine; antiarrhythmics; antiasthmatics; antidepressants; vaccines; peptides or prodrugs; hormones; proton pump inhibitors; H2 receptor antragonists; fragrances; sensates; pigments; dyes; lakes; colorants; and mixtures thereof.

21. A denture adhesive removal system comprising a wipe, wherein the wipe comprises a flexible substrate that is premoistened with a denture adhesive solvent composition.

22. The denture adhesive removal system of claim 21 wherein the flexible substrate is premoistened with a denture adhesive solvent composition comprising at least one oil-based solvent, a thickener, and a surfactant.

23. The denture adhesive removal system of claim 21 wherein the flexible substrate is premoistened with a denture adhesive solvent composition comprising from about 1% to about 90% mineral oil; from about 1% to about 30% of a carrier; from about 0.1% to about 15% of a surfactant; from about 0.1% to about 50% of a thickener.

24. A method of removing denture adhesive and denture adhesive residue from a denture surface or from any natural surface of the oral cavity of an individual by applying a denture adhesive solvent composition comprising:
a.) at least one oil-based solvent;
b.) a thickener; and
c.) a surfactant, to the denture adhesive or the denture adhesive residue.

25. (canceled)

26. The method of claim 24 wherein the denture adhesive solvent composition is applied to the denture surface or natural surface of the oral cavity by wiping said denture surface or said natural surface with a wipe comprising a flexible substrate that is premoistened with a denture adhesive solvent composition.

27. A kit comprising:
a.) a denture adhesive solvent composition;
b.) an applicator; and
c.) instructions for use.

28. (canceled)

29. (canceled)

30. (canceled)

31. (canceled)

32. (canceled)

33. (canceled)
